(19) United States
(12) Patent Application Publication
(10) Pub. No.: US 2021/0356447 A1
(43) Pub. Date: Nov. 18, 2021

(54) EFFICACY EVALUATION METHOD OF DRUG FOR REVERSING TUMOR MULTIDRUG RESISTANCE

(71) Applicant: JINAN UNIVERSITY, Guangdong (CN)

(72) Inventors: Yu CAI, Guangdong (CN); Bingyue WANG, Guangdong (CN); Qianwen LI, Guangdong (CN); Qingqing HUANG, Guangdong (CN)

(73) Assignee: JINAN UNIVERSITY, Guangdong (CN)

(21) Appl. No.: 16/484,463

(22) PCT Filed: Aug. 3, 2018
(86) PCT No.: PCT/CN2018/098414

§ 371 Date: Aug. 8, 2019

(51) Int. Cl. G01N 30/88; G01N 30/72; G01N 33/493

(52) U.S. Cl. CPC G01N 30/88; G01N 2030/027; G01N 33/493; G01N 30/7233

(57) ABSTRACT

According to the application, by measuring mass spectra of urine samples and/or serum samples in a healthy group, a negative control group, a positive control group and a treatment group of a drug to be evaluated, the mass spectra of the samples of the treatment group of the drug to be evaluated and the mass spectra of the samples of the negative control group are compared to calculate a difference between peak areas of characteristic fragment particle peaks of metabolic markers in the samples of the treatment group of the drug to be evaluated and in the samples of the negative control group, and a difference value is used to evaluate multidrug resistance of the drug to be evaluated. The larger the difference value is, the larger a callback value is, and the better an effect of reversing the tumor multidrug resistance is.

### BACKGROUND

### Technical Field

The present application relates to the technical field of pharmacodynamic evaluation, and more particularly, to an efficacy evaluation method of a drug for reversing tumor multidrug resistance.

### Description of Related Art

A large number of studies have shown that one of the main reasons for a low cure rate of a tumor which is caused by a poor effect of a chemotherapeutic drug is that the chemotherapeutic drug in cells is pumped out by drug resistance of tumor cells, so that an effective therapeutic dose reaching a focus location is reduced, and an ideal therapeutic effect cannot be achieved. Therefore, it is an urgent problem to be solved to find a drug for effectively reversing tumor multidrug resistance in the field of tumor therapy at present. A metabonomics technology is to monitor changes in body substances and functions caused by exogenous disturbance through changes in metabolic products in real time, with characteristics of integrity, timeliness and dynamics. At present, most cancer researchers use the metabonomics technology to find metabolic markers of early cancer and use these metabolic markers as targeted substances for diagnosing early liver cancer. For example, the Chinese patent application (Publication No.: 106153739A), “Model for Diagnosing Liver Cancer Based on Metabonomics Technology” is used to detect peripheral serum samples of early liver cancer patients and normal people by using the metabonomics technology. A diagnostic model is established by a difference of contents of corresponding metabolites between early liver cancer patients and normal people. As long as concentrations of five corresponding metabolites in detected human serum are compared and analyzed with the model of the present application, the model can be preliminarily used for diagnosing early liver cancer. However, except for direct diagnosis of diseases, it is equally important to evaluate an efficacy of a drug, and accurate evaluation of the efficacy of the drug is conducive to the development of a new drug and the guarantee of the efficacy of the drug. How to accurately evaluate an efficacy of a drug for reversing tumor multidrug resistance is one of the keys to find the drug for reversing tumor multidrug resistance.

### SUMMARY

Aiming at the above problems in the prior art, the present application provides a method capable of accurately and effectively evaluating an efficacy of a drug for reversing tumor multidrug resistance.

In order to achieve the object above, the following technical solutions are adopted in the present application.

An efficacy evaluation method of a drug for reversing tumor multidrug resistance includes the following steps of:

S1. measuring mass spectra of standard substances of metabolic markers to determine detection conditions of the metabolic markers, measuring a series of linear concentration samples of the standard substance of each metabolic marker to obtain a relationship between a peak area of a characteristic fragment particle peak and a concentration of each metabolic marker, and performing linear regression to obtain a linear regression equation, the metabolic marker including a serum metabolic marker and/or a urine metabolic marker;

the serum metabolic marker including at least one of the following substances: a-linolenic acid, 3-methylglutaric acid and 17-hydroxystearic acid, and

the urine metabolic marker including at least one of the following substances: xanthurenic acid, indole-2-carboxylic acid and 3-furoic acid;

S2. measuring mass spectra of serum samples and/or urine samples of a negative control group, a positive control group, a treatment group of a drug to be evaluated of a drug-resistant liver cancer HepG2 cell nude mouse model and a healthy group using the detection conditions determined in the step S1, wherein:

only normal saline is administrated to the healthy group once every three days for a total of seven times, with a dose of 0.2 ml each time by an administration method of tail vein injection, and the healthy group refers to healthy nude mice without being inoculated with any tumor cells;

only normal saline is administrated to the negative control group once every three days for a total of seven times, with a dose of 0.2 ml each time by an administration method of tail vein injection;

only doxorubicin is administrated to the positive control group once every three days for a total of seven times, with an administration dose of 3 mg/kg and 0.2 ml each time by an administration method of tail vein injection; and

only the drug to be evaluated is given to the treatment group of the drug to be evaluated once every three days for a total of seven times, with an administration dose of 3 mg/kg and 0.2 ml each time by an administration method of tail vein injection;

S3. comparing the mass spectra of the serum samples and/or the urine samples of each group with the mass spectra of the standard substances of the metabolic markers respectively to obtain the peak areas of the characteristic fragment particle peaks of the metabolic markers in the serum samples and/or the urine samples of each group; and

S4. determining the concentration of each metabolic marker according to the peak area of the characteristic fragment particle peak of each metabolic marker and the corresponding linear regression equation, then calculating a callback rate of the treatment group of the drug to be evaluated according to the concentration, and evaluating an efficacy of the drug for reversing tumor multidrug resistance according to the callback rate, wherein the larger the callback rate is, the better the efficacy of the drug for reversing tumor multidrug resistance to be evaluated is;

$$callback rate = \frac{F 0 - F 1}{F 0} \times 100 \%$$

F0 being a difference value between the concentration of the metabolic marker of the negative control group and a concentration of a metabolic marker of the healthy group; and

F1 being a difference value between the concentration of the metabolic marker of the treatment group of the drug to be evaluated or the positive control group and the concentration of the metabolic marker of the healthy group.

The following method can also be used in the step S4 mentioned above: respectively comparing difference values of the peak areas of the characteristic fragment particle peaks of the metabolic markers in the serum samples and/or the urine samples of the positive control group, the treatment group of the drug to be evaluated and the negative control group, and reflecting the concentration of each metabolic marker in each group by the difference value of the peak areas, so as to evaluate the efficacy of the drug for reversing tumor multidrug resistance to be evaluated, wherein the larger the difference value of the concentrations is, the better the efficacy of the drug for reversing tumor multidrug resistance to be evaluated is.

Compared with the prior art, the present application has the beneficial effects that: according to the present application, the pharmacodynamics of the drug for reversing tumor cell multidrug resistance to be evaluated is evaluated by the concentration difference value through detecting the concentrations of the metabolic markers in the urine samples and/or the serum samples of the negative control group, the positive control group and the treatment group of the drug to be evaluated, and the larger the difference value is, the larger the callback value is, and the better the effect of reversing tumor multidrug resistance is. By experimental verification, the method according to the application can accurately evaluate an efficacy of the drug for reversing tumor multidrug resistance, and the method according to the application can also be applied to early diagnosis of drug resistance generated by liver cancer cells.

### DESCRIPTION OF THE EMBODIMENTS

In order to fully understand the technical contents of the present application, the technical solutions of the present application are further introduced and explained below with reference to the detailed embodiments.

Implementation Method:

Liver cancer HepG2/ADR tumor-transplanted nude mouse models were firstly established and divided into four groups with 30 mice in each group, and after one week of successful establishment of the model, the mice were administered in different methods:

in a healthy group, 30 healthy nude mice without being inoculated with any tumor cells were administrated with normal saline only;

in a negative control group, 30 mice were administrated with normal saline only;

in a positive control group, 30 mice were administrated with doxorubicin (DOX) only;

in a treatment group 1 of a drug to be evaluated, 30 mice were administrated with a crude drug of doxorubicin and a crude drug of psoralen; and

in a treatment group 2 of a drug to be evaluated, 30 mice were administrated with the crude drug of doxorubicin and psoralen polymer lipid nanoparticles.

Metabolic markers were quantified by a targeted metabonomics technology, and an efficacy of a drug for reversing tumor multidrug resistance to be evaluated was evaluated by a concentration of each metabolic marker.

A preparation method of the psoralen polymer lipid nanoparticles was as follows.

Preparation of an Emulsifier Solution:

preparation of Tween 80 solution (35 mg/ml, diluted with water): 20 ml of Tween 80 was taken and added with 160 ml of water, and the mixture was stirred evenly to obtain an emulsifier solution with a concentration of 35 mg/ml as a first standby solution.

Water Phase:

1,2-distearoyl-sn-glycero-3-phosphoethanolamine-N-[methoxy(polyethylene glycol)-2000] (DSPE-PEG2000) was weighed and dissolved in ethanol to prepare a DSPE-PEG2000 solution with a concentration of 5 mg/ml.

Phosphatidylcholine (PC) was weighed and dissolved in ethanol to prepare a PC solution with a concentration of 40 mg/ml.

9 ml of the DSPE-PEG2000 solution and 6.38 ml of the PC solution mentioned above were stirred evenly and then added into the first standby solution, and the mixture was heated and mixed evenly at 70° C. for 2 to 3 minutes to be used as a second standby solution.

Oil Phase:

Psoralen was weighed and dissolved in acetonitrile to prepare a psoralen solution with a concentration of 1.5 mg/ml.

Polylactic acid-glycolic acid copolymer (PLGA) was weighed and dissolved in acetonitrile to prepare a PLGA solution with a concentration of 10 mg/ml.

10 ml of the psoralen solution and 10 ml of the PLGA solution mentioned above were fully mixed, then an oil phase solution was slowly added into the second standby solution with an injector, the mixture was continuously heated and stirred at 70° C. for 90 minutes, and then cooled to a room temperature, and after sterile filtering, the psoralen polymer lipid nanoparticles were obtained.

The detailed implementation methods are as follows.

I. Construction of Liver Cancer HepG2/ADR Tumor-Transplanted Nude Mouse Model

Cell thawing: a proper amount of HepG2/ADR tumor cells frozen in a liquid nitrogen tank were taken out and quickly placed in a 37° C. water bath and continuously shaken, so as to quickly thaw out and pass a dangerous period. A complete medium was added to dilute a cryoprotectant DMSO in a cell cryopreservation tube to reduce toxicity, centrifugation (1000 rpm) was performed for 5 minutes, a supernatant was discarded, and the complete medium was added again, and cultured in a 5% CO₂ cell incubator at 37° C.

Cell passage: after the thawed cells were cultured for 24 hours, a culture solution in a culture vessel was discarded. Residual serum in the culture vessel was removed by washing once with PBS, 1 ml of pancreatin was added for digestion at 37° C. for 30 seconds to 2 minutes, and when the cells could be easily blown off by a pipette tip, a complete medium containing serum was quickly added to prevent an enzymatic hydrolysis reaction. A wall of the cell culture vessel was continuously blown by the pipette tip to blow off the adherent cells, centrifugation (1000 rpm) was performed for 5 minutes, a supernatant was discarded, a fresh complete medium was added, after being evenly dispersed by the pipette tip, the cells were divided into new cell culture vessels (preferably 1:4 to 1:6), and a cell culture solution was changed twice a week during passage.

Cell collecting and counting: when the cells were cultured to a certain number, a vessel of cells were collected, 180 μl of cell sap was added with 20 μl of PBS, after mixing evenly, 10 μl of the mixture was placed on a glass slide, during which bubble generation should be carefully avoided, the glass slide was placed under the microscope for counting, a cell concentration was counted, and then the required cells were collected and diluted to a concentration required for final transplantation.

Cell transplanting: ten nude mice without thymus, including five male mice and five female mice, were selected, after 10-day adaptation period, 0.2 ml of HepG2/ADR cells (cell concentration: 6×10 7 cells/ml) were subcutaneously injected into each nude mouse with a 23G sterile needle, and in order to prevent tumor cells from being destroyed and matrix gel from being solidified in the period, the injection should be completed as soon as possible. Before injection, the needle was shaken left and right to form an expanded subcutaneous cyst (because if the injection was performed to a specific area by not shaking the needle, the mixture could form a large cell mass, and ineffective perfusion of nutrients in a core of the mass into the cells could result in subsequent growth defects), and when the needle was taken out, the injector was rotated to prevent leakage of cell sap. After subcutaneous injection, the matrix gel was irreversibly solidified to form a red bulge, about 10 days later, the solidified matrix gel was eliminated, and the tumor cells were successfully inoculated, and after a tumor mass grew for three weeks, nude mice with the tumor of the same size (about 7 mm in diameter) were selected. The subcutaneous tumor mass was taken out, a peripheral part of the tumor mass was divided into small tumor masses of 2×2×2 mm, after the nude mice (nu/nu-BALB/c, half male nude mice and half female nude mice at six weeks old, n=30) were anesthetized with isoflurane, the small tumor masses were subcutaneously implanted into shoulders of the nude mice to make the tumor masses of liver cancer model mice similar in size and shape, thus reducing experimental errors caused by different tumor mass sizes. After successful transplantation, growth statuses of the model nude mice were observed and recorded every day, weights of the nude mice were recorded every two days, and tumor sizes (length and width) were measured with a vernier caliper.

II. Finding of Differential Metabolites by Non-Targeted Metabonomics and Main Metabolic Pathway Involved

Collection of urine: at least 0.5 ml of urine of 10 healthy nude mice (blank) and 10 liver cancer model nude mice was respectively collected with a mouse metabolic cage, and placed in a centrifuge tube with low protein adsorption of 1.5 ml, 10 μl of preservative solution with a mass volume fraction of 1% was added, centrifugation was performed at a low temperature (4° C., 3000 g/min) for 10 minutes, and a supernatant was gently sucked into a precooled labeled centrifuge tube, and cryopreserved at 80° C. below zero for later use.

Collection of serum: after anaesthetizing the nude mice in the 2.2.1.1 above with a proper amount of anaesthetic, 0.5 ml to 1 ml of blood was collected by a cardiac blood collection method, after standing at a room temperature for 30 minutes, centrifugation was immediately performed twice (3000 rpm/8000 rpm, 4° C., 10 minutes each time), and about 200 μl of supernatant serum was taken and stored at 80° C. below zero for later use.

Collection of serum: after anaesthetizing the nude mice mentioned above with anaesthetic, 0.5 ml to 1 ml of blood was collected by a cardiac blood collection method, after standing at a room temperature for 30 minutes, centrifugation was immediately performed twice (3000 rpm/8000 rpm, 4° C., 10 minutes each time), and about 200 μl of supernatant serum was taken and stored at 80° C. below zero for later use.

Preprocessing of Urine:

1. 150 μL of urine was centrifuged at a low temperature for 10 minutes (13000 rpm, 4° C.), 100 μL of supernatant was taken and transferred into a 1.5 ml EP tube, 10 μL of internal standard (L-2-chlorophenylalanine, 0.3 mg/mL, methanol configured) was added, and subjected to vortex shaking for 10 seconds;

2. 100 μL of methanol-acetonitrile (2:1, v/v) was added, and subjected to vortex shaking for 1 minute;

3. ultrasonic extraction was performed in an ice-water bath for 10 minutes;

4. still standing was performed at 20° C. below zero for 30 minutes;

5. centrifugation was performed for 15 minutes (13000 rpm, 4° C.), 150 μL of supernatant was sucked with an injector, filtered with a 0.22 μm organic phase pinhole filter, transferred into a LC injection vial, and stored at 80° C. below zero until LC-MS analysis was performed; and

6. quality control samples (QC) were prepared by mixing all urine samples in equal volumes, and an injection volume of each QC was the same as that of other urine samples.

Note: all reagents should be precooled at 20° C. below zero before use.

Preprocessing of Serum:

1. 100 μL of serum was add with 10 μL of internal standard (L-2-chlorophenylalanine, 0.3 mg/mL, methanol configured), and subjected to vortex shaking for 10 seconds;

2. 300 μL of protein precipitant methanol-acetonitrile (2:1, v/v) was added, and subjected to vortex shaking for 1 minute;

3. ultrasonic extraction was performed in an ice-water bath for 10 minutes;

4. still standing was performed at 20° C. below zero for 30 minutes;

5. centrifugation was performed for 15 minutes (13000 rpm, 4° C.), 200 μL of supernatant was sucked with an injector, filtered with a 0.22 μm organic phase pinhole filter, transferred into a LC injection vial, and stored at 80° C. below zero until LC-MS analysis was performed; and

6. quality control samples (QC) were prepared by mixing all serum samples in equal volumes, and an injection volume of each QC was the same as that of other serum samples.

Note: all reagents should be precooled at 20° C. below zero before use.

III. Measurement of Mass Spectra of Urine and Serum Samples of Liver Cancer HepG2/ADR Tumor-Transplanted Nude Mouse Model

Mass spectra of the urine sample, the serum sample and the quality control sample of each liver cancer HepG2/ADR tumor-transplanted nude mouse were measured respectively, and original LC-MS spectra of the samples were collected. An analytical instrument in the embodiment was a liquid chromatography-mass system composed of a Waters' I-Class ultra-high performance liquid chromatography and a VION IMS QT of high-resolution mass spectrometer in series.

Chromatographic conditions were as follows:

chromatographic column: ACQUITY UPLC BEH C18 (100 mm×2.1 mm, 1.7 μm);

column temperature: 45° C.;

mobile phase: phase A: water (containing 0.1% formic acid), phase B: is acetonitrile/methanol (2/3) (v/v) (containing 0.1% formic acid);

flow speed: 0.4 mL/min; and

injection volume: 1 μL.

Volume percentages of phase A and phase B of an eluent during gradient elution (calculated from a sample injection time) were as follows:

| Time/min | 0 | 1 | 2.5 | 6.5 | 8.5 | 10.7 | 10.8 | 13 |
|---|---|---|---|---|---|---|---|---|
| A % | 99 | 70 | 40 | 10 | 0 | 0 | 99 | 99 |
| B % | 1 | 30 | 60 | 90 | 100 | 100 | 1 | 1 |

Mass-spectrometric conditions were as follows:

ion source: ESI;

positive and negative ion scanning modes were used to collect mass-spectrometric signals of each sample respectively;

| parameter | positive/negative ion; |
|---|---|
| electrospray capillary voltage (kV) | 2.5; |
| injection voltage (DP, V) | 40; |
| collision voltage (CE, eV) | 6; |
| iron source temperature (° C.) | 115 |
| desolvation temperature (° C.) | 450; |
| desolvation gas flow (L/h) | 900; |
| mass range (amu) | 50 to 1000; |
| scan time (s) | 0.2; and |
| interscan delay (s) | 0.02. |

One QC sample was inserted into every eight analysis samples, and the stability and reproducibility of a whole analysis process were investigated through the triable inspection of a base peak chromatogram.

IV. Screening and Determination of Metabolic Markers in Serum and Urine Samples

The original LC-MS chromatograms of the urine, serum and quality control samples were respectively subjected to baseline filtering, peak identification, integration, retention time correction, peak alignment and normalization via a metabonomics processing software, progenesis QI (Waters Corporation, Milford, USA), to obtain a data matrix of a retention time, a mass-to-charge ratio and a peak intensity.

Multivariate statistical analysis: the obtained data matrix was subjected to multivariate statistical analysis (SIMCA-P software package, Version 14.1, Umetrics, Umeå, Sweden), and after ID and other information were all set, unsupervised principal component analysis (PCA) was firstly performed to investigate an overall distribution status of samples of the healthy group and the liver cancer model group to determine whether there was a trend of separation. Meanwhile, the stability of a whole analysis system and the reliability of sample preprocessing were investigated by observing the proximity and overlap degree of the QC samples, then supervised (orthogonal) partial least square discriminant analysis was established to distinguish different metabolic profiles between the two groups of samples, the overall difference was observed through the metabolic profiles between the two groups, and finally, in order to prevent the over-fitting of the model under a supervised status, an internal verification method (200 response sequencing tests and seven cycles of interactive verification) was used to examine the quality of the model.

Screening, searching and nature determination of differential metabolites: a method of multi-dimensional and single-dimensional combination was used to screen the differential metabolites between the two groups of samples, and the specific method was as follows:

Multi-dimensional method (selected from a statistical chart): a metabolite far from a central point in an s-plots load diagram, a metabolite with a high pq value on a first principal component in a Loading histogram, a variable with a VIP value of the first principal component greater than 1 in an OPLS-DA model, and a metabolite with a higher −log 10 (P-value) value when a difference multiple was 2 in a Volcano Plot were included; meanwhile, a heat map was used to check an approximate content change for verification.

One-dimensional method: differential metabolites (p<0.05) with statistical significance were inspected by T.

Searching and nature determination: natures of the selected differential metabolites were determined using a QI database of waters. The metabolic markers in the serum sample and the urine sample were shown in the following table:

metabolic makers in serum sample: a-linolenic acid, 3-methylglutaric acid and 17-hydroxystearic acid;

metabolic makers in urine sample: xanthurenic acid, indole-2-carboxylic acid and 3-furoic acid.

The method mentioned above was used, and a sample quantity was increased to be screened again, so as to verify the metabolic markers above. Metabolic markers screened again were the same as the metabolic markers mentioned above.

V. Establishment of Liver Cancer Drug-Resistant Nude Mouse Model

Healthy group: only normal saline was administrated to 30 healthy nude mice without being inoculated with any tumor cells once every three days for a total of seven times, with a dose of 0.2 ml each time by an administration method of tail vein injection.

Negative control group: only normal saline was administrated to 30 liver cancer HepG2/ADR tumor-transplanted nude mouse models once every three days for a total of seven times, with a dose of 0.2 ml each time by an administration method of tail vein injection.

Positive control group: only doxorubicin was administrated to 30 liver cancer HepG2/ADR tumor-transplanted nude mouse models once every three days for a total of seven times, with a dose of 3 mg/kg and 0.2 ml each time by an administration method of tail vein injection.

Treatment group 1 of a drug to be evaluated: a crude drug of doxorubicin and a crude drug of psoralen were administrated to 30 liver cancer HepG2/ADR tumor-transplanted nude mouse models once every three days for a total of seven times, with a dose of 3 mg/kg and 0.2 ml each time by an administration method of tail vein injection.

Treatment group 2 of a drug to be evaluated: crude drugs of doxorubicin and psoralen polymer lipid nanoparticles were administrated to 30 liver cancer HepG2/ADR tumor-transplanted nude mouse models once every three days for a total of seven times, with a dose of 3 mg/kg and 0.2 ml each time by an administration method of tail vein injection.

VI. Collection and Preprocessing of Serum Sample and Urine Sample

The collection and preprocessing methods of serum and urine were the same as those described in “II. Finding of differential metabolites by non-targeted metabonomics and main metabolic pathway involved”.

VII. Measurement of Mass Spectrum of Standard Substance of Metabolic Marker

Mass spectra of standard substances of a-linolenic acid, 3-methylglutaric acid, 17-hydroxystearic acid, xanthurenic acid, indole-2-carboxylic acid and 3-furoic acid were measured to determine mass-spectrometric detection conditions of each metabolic marker. The detection conditions used in the embodiment were as follows:

detection instrument: AB SCIEX 3500 QQQ triple quadrupole liquid chromatography-mass instrument.

Liquid-phase conditions were as follows:

chromatographic column: WATERS ACQUITYUPLC BEH C18 (100 mm×2.1 mm, 1.7 μm);

column temperature: 45° C.;

injection volume: 5 μL;

mobile phase: A: aqueous solution containing 0.1% formic acid; B: mixed solution of acetonitrile (containing 0.1% formic acid) and methanol in a volume ratio of 2:3; and

flow speed: 0.3 mL/min.

Volume percentages of phase A and phase B of an eluent during gradient elution (calculated from a sample injection time) were as follows:

| Time/min | 0 | 2 | 5 | 7 | 9 | 13 | 15 |
|---|---|---|---|---|---|---|---|
| A % | 95 | 70 | 35 | 10 | 10 | 95 | 95 |
| B % | 5 | 30 | 65 | 90 | 90 | 5 | 5 |

Mass-spectrometric conditions were as follows:

a multi-reaction detection mode was used, desolvation gas and conical hole blowback gas were nitrogen, and the flow rates were 800 mL/min and 50 ml/min respectively;

ion source: ESI⁺; and

ion source parameters: curtain gas: 30 psi; GS1: 55 psi; and GS2: 60 psi.

MRM parameters were as follows:

| | Parent | Daughter ion (m/z) | | | |
|---|---|---|---|---|---|
| | ion | Qualitative | Quantitative | DP | CE |
| Metabolic marker | (m/z) | daughter ion | daughter ion | value | value |
| A-linolenic acid | 279 | 90 | 108 | 70 | 247 |
| 3-methylglutaric | 145.13 | 68 | 100.4 | 60 | 69 |
| acid | | | | | |
| 17-hydroxystearic | 302.48 | 60 | 197 | 75 | 55 |
| acid | | | | | |
| Xanthurenic acid | 207.17 | 85 | 193 | 60 | 165 |
| indole-2-carboxylic | 163.16 | 52 | 141 | 70 | 115 |
| acid | | | | | |
| 3-furoic acid | 113.08 | 55 | 99 | 60 | 87 |

A series of linear concentration samples of the standard substance of each metabolic marker were respectively measured using the detection conditions above to obtain a relationship between a peak area of a characteristic fragment particle peak and a concentration of each metabolic marker, and linear regression was performed to obtain a linear regression equation.

VII. Detection of Mass Spectra of Serum Samples and Urine Samples of Each Group

Mass spectra of serum samples and urine samples of the positive control group, the negative control group, the treatment group 1 of a drug to be evaluated and the treatment group 2 of a drug to be evaluated of a drug-resistant liver cancer HepG2 cell nude mouse model were measured to obtain the peak areas of the characteristic fragment particle peaks of the metabolic markers in the serum samples and the urine samples of each group. The detection and analysis instruments and the detection conditions were the same as those in “VII. Measurement of mass spectrum of standard substance of metabolic marker”.

VIII. Analysis and Evaluation

The concentration of each metabolic marker was determined according to the peak area of the characteristic fragment particle peak of each metabolic marker and the corresponding linear regression equation (the concentration of the healthy group was C1, the concentration of the negative control group was C2, the concentration of the positive control group was C3, the concentration of the treatment group 1 was C4, and the concentration of the treatment group 2 was C5), then callback rates of the positive control group, the negative control group, the treatment group 1 of the drug to be evaluated and the treatment group 2 of the drug to be evaluated were calculated according to the concentrations, and an efficacy of the drug for reversing tumor multidrug resistance was evaluated according to the callback rate, wherein the larger the callback rate was, the better the efficacy of the drug for reversing tumor multidrug resistance to be evaluated was.

$$Callback rate = \frac{F 0 - F 1}{F 0} \times 100 \%$$

F0 was a difference value between the C2 of the negative control group and the C1 of the healthy group.

F1 was a difference value between the C3 of the positive control group/the C4 of the treatment group 1 of the drug to be evaluated/the C5 of the treatment group 2 of the drug to be evaluated and the C1 of the healthy group.

The results were shown in the following table:

| | | | | Treatment | Treatment |
|---|---|---|---|---|---|
| | | Negative | Positive | group 1 of | group 2 of |
| | Healthy | control | control | drug to be | drug to be |
| | group | group | group | evaluated | evaluated |
| | Callback | Callback | Callback | Callback | Callback |
| Metabolic marker | rate | rate % | rate % | rate % | rate % |
| A-linolenic acid | / | 0 | 17.56 | 48.72 | 53.64 |
| Xanthurenic acid | / | 0 | 19.08 | 23.78 | 41.25 |
| 3-methylglutaric | / | 0 | 13.69 | 25.37 | 39.82 |
| acid | | | | | |
| 17-hydroxystearic | / | 0 | 15.54 | 29.49 | 42.47 |
| acid | | | | | |
| indole-2-carboxylic | / | 0 | 12.73 | 23.17 | 38.11 |
| acid | | | | | |
| 3-furoic acid | / | 0 | 12.30 | 21.12 | 47.50 |

It can be seen from the callback rate of each group in the table above that the callback rate of the treatment group 2 of the drug to be evaluated is the largest, followed by the callback rate of the treatment group 1 of the drug to be evaluated. Therefore, it can be judged that the effect of the treatment group 2 of the drug to be evaluated is significantly better than the effect of combined administration of crude drugs of doxorubicin and crude drugs of psoralen, which indicates that the psoralen really has the effect of reversing tumor drug resistance, and the reversibility is better after being prepared into polymer nanoparticles.

In addition, difference values of the peak areas of the characteristic fragment particle peaks of the metabolic markers in the serum samples and/or the urine samples of the positive control group, the treatment group 1 of the drug to be evaluated, the treatment group 2 of the drug to be evaluated and the negative control group are respectively compared, and the concentration of each metabolic marker in each group is reflected by the difference value of the peak areas, so as to evaluate the efficacy of the drug for reversing tumor multidrug resistance to be evaluated, wherein the larger the difference value of the concentrations is, the better the efficacy of the drug for reversing tumor multidrug resistance to be evaluated is.

The description above further illustrates the technical content of the application by the embodiments only, so that readers can understand the invention more easily, but it doesn't mean that the implementation of the invention is limited thereto. Any technical extension or recreation made based on the invention is protected by the invention.

1. An efficacy evaluation method of a drug for reversing tumor multidrug resistance, comprising the following steps of:
S1, measuring mass spectra of standard substances of metabolic markers to determine detection conditions of the metabolic markers, measuring a series of linear concentration samples of the standard substance of each metabolic marker to obtain a relationship between a peak area of a characteristic fragment particle peak and a concentration of each metabolic marker, and performing linear regression to obtain a linear regression equation, the metabolic marker comprising a serum metabolic marker and/or a urine metabolic marker,
the serum metabolic marker comprising at least one of the following substances: α-linolenic acid, 3-methylglutaric acid and 17-hydroxystearic acid,
the urine metabolic marker comprising at least one of the following substances: xanthurenic acid, indole-2-carboxylic acid and 3-furoic acid;
S2, measuring mass spectra of serum samples and/or urine samples of a positive control group, a negative control group and a treatment group of a drug to be evaluated of a drug-resistant liver cancer HepG2 cell nude mouse model using the detection conditions determined in the step S1;
S3, comparing the mass spectra of the serum samples and/or the urine samples of each group with the mass spectra of the standard substances of the metabolic markers respectively to obtain peak areas of characteristic fragment particle peaks of the metabolic markers in the serum samples and/or the urine samples of each group; and
S4, evaluating through a first evaluation method or a second evaluation method;
the first evaluation method being as follows: determining a concentration of each metabolic marker according to the peak area of the characteristic fragment particle peak of each metabolic marker and the corresponding linear regression equation, then calculating a callback rate of the treatment group of the drug to be evaluated according to the concentration, and evaluating an efficacy of the drug for reversing tumor multidrug resistance according to the callback rate, wherein the larger the callback rate is, the better the efficacy of the drug for reversing tumor multidrug resistance to be evaluated is,
callback rate=(F0−F1)/F0×100%
F0 being a difference value between the concentration of the metabolic marker of the negative control group and a concentration of a metabolic marker of a healthy group,
F1 being a difference value between the concentration of the metabolic marker of the treatment group of the drug to be evaluated and the concentration of the metabolic marker of the healthy group,
the second evaluation method being as follows: respectively comparing difference values of the peak areas of the characteristic fragment particle peaks of the metabolic markers in the serum samples and/or the urine samples of the positive control group, the treatment group of the drug to be evaluated and the negative control group, and reflecting a concentration of each metabolic marker in each group by the difference value of the peak areas, so as to evaluate the efficacy of the drug for reversing tumor multidrug resistance to be evaluated, wherein the larger the difference value of the concentrations is, the better the efficacy of the drug for reversing tumor multidrug resistance to be evaluated is.

2. The efficacy evaluation method of the drug for reversing tumor multidrug resistance according to claim 1, wherein only doxorubicin is administrated to the positive control group once every three days for a total of seven times, with an administration dose of 3 mg/kg and 0.2 ml each time by an administration method of tail vein injection.

3. The efficacy evaluation method of the drug for reversing tumor multidrug resistance according to claim 1, wherein only normal saline is administrated to the negative control group once every three days for a total of seven times, with 0.2 ml each time by an administration method of tail vein injection.

4. The efficacy evaluation method of the drug for reversing tumor multidrug resistance according to claim 1, wherein the drug to be evaluated is administrated to the treatment group of the drug to be evaluated once every three days for a total of seven times, with an administration dose of 3 mg/kg and 0.2 ml each time by an administration method of tail vein injection.
